(19) United States
(12) Patent Application Publication
(10) Pub. No.: US 2024/0245905 A1
(43) Pub. Date: Jul. 25, 2024

(54) SYSTEM AND METHOD FOR STIMULATING A NERVE, AUTOMATED RESUSCITATION SYSTEM

(71) Applicant: Oxford University Innovation Limited, Oxford (GB)

(72) Inventors: James FITZGERALD, Oxford (Oxfordshire) (GB); Brian ANDREWS, Oxford (Oxfordshire) (GB); Adrian POULTON, Oxford (Oxfordshire) (GB); Jonathan JARVIS, Oxford (Oxfordshire) (GB); Francisco SAAVEDRA, Oxford (Oxfordshire) (GB); Conor KEOGH, Oxford (Oxfordshire) (GB)

(21) Appl. No.: 18/564,140

(22) PCT Filed: May 26, 2022
(86) PCT No.: PCT/GB2022/051345

§ 371 Date: Nov. 27, 2023

(30) Foreign Application Priority Data
May 28, 2021 (GB) 2107670.8

(51) Int. Cl. A61N 1/04; A61N 1/36; A61N 1/39

(52) U.S. Cl. CPC A61N 1/0456; A61N 1/0492; A61N 1/36031; A61N 1/36034; A61N 1/39044; A61N 1/3993

(57) ABSTRACT

Methods and systems are disclosed for electrically stimulating a nerve to cause muscle contraction. In one arrangement, a plurality of electrodes is positioned in contact with skin. The electrodes allow electrical stimulation to be directed independently towards a plurality of different stimulation regions below the skin via a corresponding plurality of combinations of the electrodes. For each combination of electrodes, a test stimulation is applied using the combination of electrodes and a response to the test stimulation is measured. One or a subset of stimulation regions is selected based on the measured responses. A therapy stimulation is applied to each selected stimulation region using the combination of electrodes corresponding to the selected stimulation region.

The present disclosure relates to stimulating nerves in order to cause muscle contraction and is particularly applicable to stimulating phrenic nerves to maintain diaphragm health during a period of assisting ventilation, or as part of an automated resuscitation system.

In normal breathing, electrical signals from the brain are sent to the diaphragm via the phrenic nerves to cause the diaphragm to contract and thus expand the lungs. In medical situations where breathing is assisted by an external mechanical ventilator, the reduced demand on the diaphragm can cause undesirable muscle loss in the diaphragm over time. This effect can result in a condition known as Ventilator Induced Diaphragmatic Dysfunction (VIDD).

To prevent or reverse diaphragm muscle atrophy, it is known to provide artificial electrical stimulation to the phrenic nerves. This has been done for example by intravenously inserting a catheter containing electrodes to a position where the electrodes are close to the phrenic nerves. Small electrical signals are then sent to the electrodes to stimulate contraction of the diaphragm. This action can be timed to assist breathing, in order to avoid conflict with the external ventilator. In alternative approaches, temporary electrodes may be implanted by a surgeon into the diaphragm muscle itself during surgical operations that are expected to require post-operative critical care.

The above approaches have been shown to be effective in providing adequate stimulation of the diaphragm but require significant surgical interventions. They can only be implemented in sophisticated medical settings, require availability of specialist staff, place significant demands on medical resources, and can involve a degree of stress, discomfort and/or risk for some patients, particularly those who are in a fragile condition (although stress and discomfort may not apply where patients are asleep).

It is also known to stimulate muscles using non-invasive electrical stimulation. For example, electrode systems are known for stimulating contraction of abdominal muscles for muscle toning or in the context of treating patients on mechanical ventilation to prevent disuse atrophy of abdominal wall muscles. It is not convenient to apply such approaches directly to stimulation of the diaphragm, however, because the phrenic nerves are more difficult to access and can move with movement of the head and torso. It is therefore necessary either to use surgical approaches such as those described above or allow for electrodes to be pressed firmly against the skin and/or frequently repositioned to maintain sufficiently good electrical contact between the electrodes and phrenic nerves.

It is an object of the present disclosure to provide an alternative approach for applying stimulation to nerves such as the phrenic nerves that is easier and/or quicker to apply and/or more reliable and/or less costly.

According to an aspect, there is provided a system for electrically stimulating a nerve to cause muscle contraction, comprising: a plurality of electrodes configured to be positioned in electrical contact with the skin simultaneously, the electrodes being configured to allow electrical stimulation to be directed independently towards a plurality of different stimulation regions below the skin via a corresponding plurality of combinations of the electrodes; and a controller configured to: for each combination of electrodes, apply a test stimulation using the combination of electrodes and measure a response to the test stimulation; select one or a subset of stimulation regions based on the measured responses; and apply a therapy stimulation to each selected stimulation region using the combination of electrodes corresponding to the selected stimulation region.

Thus, a system is provided which allows nerve stimulation to be achieved non-invasively, reliably and with minimal adjustment and/or supervision. The system can be deployed quickly and easily, with minimal requirements for specialist staff at the point of installation and on an ongoing basis. The approach can be used in a wide range of scenarios, including situations where subject movement is likely to disrupt optimal positioning of electrodes and/or where it is difficult or impossible to apply external pressure to electrodes to hold them firmly in place. For example, the approach is effective where movement of the head relative to the torso causes the position of the electrode relative to the target nerve to change. These changes might result in reduced physiological response. The unique combination of features of the above embodiment is achieved by providing electrodes that allow multiple different stimulation regions to be selectively stimulated using corresponding combinations of electrodes and scanning through those combinations with test stimulations and measured responses to those stimulations to select an optimum combination (or combinations) of electrodes to apply the therapy.

In some arrangements, the controller is further configured to perform an electrode contact quality check comprising measuring an electrical impedance associated with a contact between an electrode and the skin for each of the plurality of combinations of the electrodes. This feature can help ensure that the therapy stimulations are only applied where the corresponding electrodes are all in good electrical contact with the skin. Where contact between an electrode and the skin is disrupted for some reason, due to movement or degradation over time for example, that electrode can be excluded from the stimulation and/or an alert can be issued to prompt a carer to manually assess the electrodes and/or reattach or replace them as necessary.

In some arrangements, the plurality of electrodes comprises a plurality of first electrodes configured to be brought simultaneously into contact with respective different locations on the skin; and one or more second electrodes configured to contact one or more respective further locations on the skin, and to have different polarity to the first electrodes during operation of the system. The plurality of electrodes is configured such that a skin contact region of each of one or more of the second electrodes surrounds a skin contact region of one or more of the first electrodes. Providing an arrangement with second electrodes surrounding first electrodes provides a more focused and/or deeper stimulation, which may reduce the magnitude of electrical charge or current that is needed to achieve a given physiological response, particularly when combined with the use of the test stimulations to continually select and update a choice of optimal electrode combinations to use for the therapy. This approach is particularly desirable where it is desired to stimulate a nerve that is relatively deep and where it is not appropriate to press the electrodes against the skin (such as is the case for stimulation of phrenic nerves via electrodes on the neck).

The focus achieved by the concentric electrode approach (having skin contact regions of second electrodes surrounding skin contact regions of first electrodes) depends on the geometrical dimensions of the respective electrodes. These geometrical dimensions can be adjusted to achieve more or less focus and/or depth of activation according to the particular needs of the application. For example, there are safety limitations to the current density that can be applied to the skin without tissue damage. The concentric electrode approach allows greater depth of activation for a given skin current density.

In some arrangements, the plurality of electrodes is configured such that the skin contact region of each of one or more of the second electrodes surrounds the skin contact region of multiple first electrodes, preferably with at least one of the second electrodes being configured such that the skin contact regions of at least two of the first electrodes surrounded by the skin contact region of the second electrode are not separated from each other by a portion of the skin contact region of the second electrode. This configuration makes it easier to provide a high level of spatial granularity. Fine spatial separation between the centres of different stimulation regions can be achieved without sacrificing the desired spatial focusing effect provided by arranging for the first electrodes to be surrounded by a second electrode.

According to a further aspect, there is provided a system for electrically stimulating a nerve to cause muscle contraction, comprising: a plurality of electrodes configured to be positioned in electrical contact with the skin simultaneously, the electrodes being configured to allow electrical stimulation to be directed independently towards a plurality of different stimulation regions below the skin via a corresponding plurality of combinations of electrodes, wherein the plurality of electrodes comprises: a plurality of first electrodes configured to be brought simultaneously into contact with respective different locations on the skin; and one or more second electrodes configured to contact one or more respective further locations on the skin, and to have different polarity to the first electrodes during operation of the system, wherein the plurality of electrodes is configured such that a skin contact region of each of one or more of the second electrodes surrounds a skin contact region of a plurality of the first electrodes.

According to a further aspect, there is provided a method of electrically stimulating a nerve to cause muscle contraction, comprising: positioning a plurality of electrodes in electrical contact with the skin of a subject simultaneously, the electrodes being configured to allow electrical stimulation to be directed independently towards a plurality of different stimulation regions below the skin via a corresponding plurality of combinations of the electrodes; and for each combination of electrodes, applying a test stimulation using the combination of electrodes and measuring a response to the test stimulation; selecting one or a subset of stimulation regions based on the measured responses; and applying a therapy stimulation to each selected stimulation region using the combination of electrodes corresponding to the selected stimulation region.

According to a further aspect, there is provided a method of electrically stimulating a nerve to cause muscle contraction, comprising: positioning a plurality of electrodes in electrical contact with the skin of a subject simultaneously, the electrodes being configured to allow electrical stimulation to be directed independently towards a plurality of different stimulation regions below the skin via a corresponding plurality of combinations of electrodes, wherein the plurality of electrodes comprises: a plurality of first electrodes simultaneously in contact with respective different locations on the skin; and one or more second electrodes contacting one or more respective further locations on the skin, a skin contact region of each of one or more of the second electrodes surrounding a skin contact region of a plurality of the first electrodes, operating the electrodes with the one or more second electrodes having different polarity to the first electrodes.

Arrangements of the disclosure will be further described by way of example only with reference to the accompanying drawings.

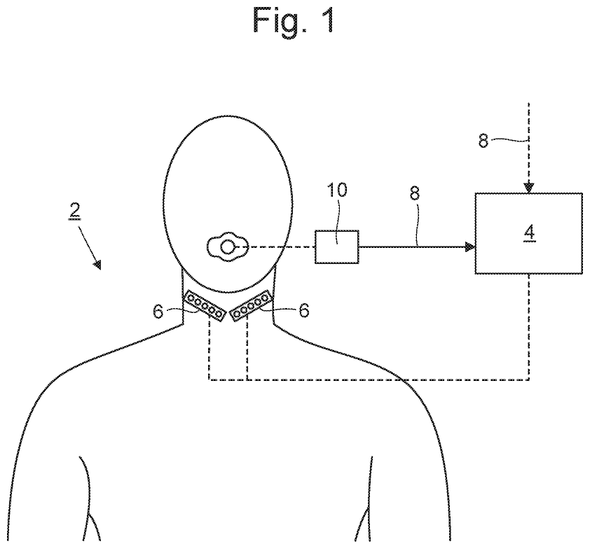

FIG. 1 schematically depicts a system for electrically stimulating a nerve applied to stimulation of phrenic nerves;

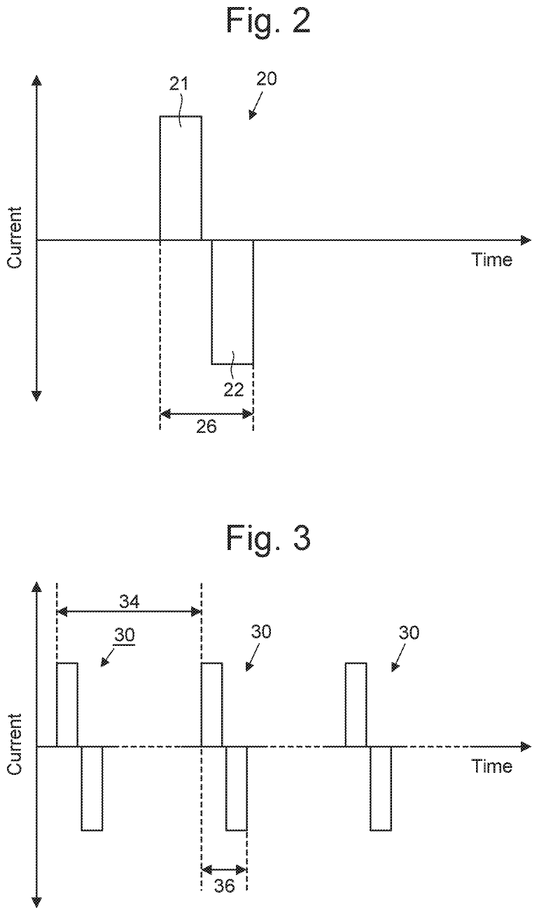

FIG. 2 is a graph of current against time to schematically depict an example test waveform of a test stimulation;

FIG. 3 is a graph of current against time to schematically depict therapy waveforms of an example therapy stimulation;

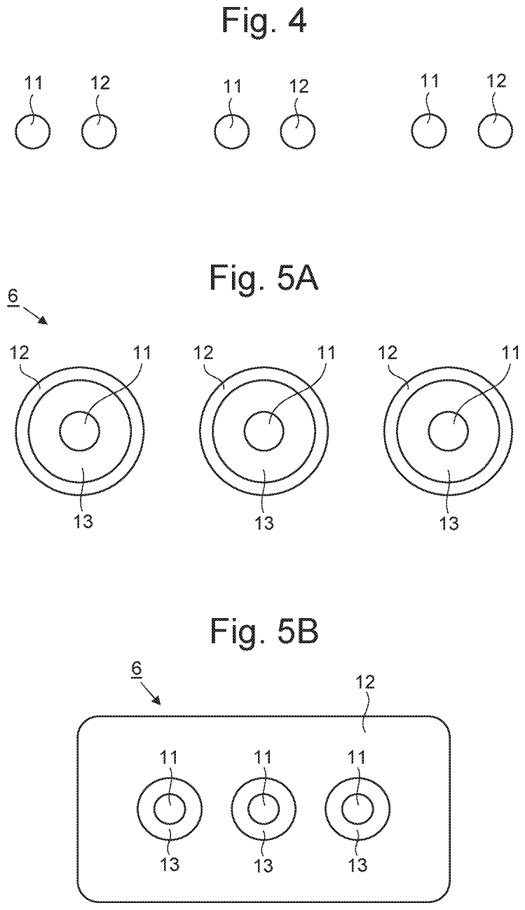

FIG. 4 depicts example electrodes provided in bipolar pairs of identical electrodes (viewed perpendicularly to a skin contact region corresponding to the electrodes);

FIG. 5A depict further example electrodes having pairs in which a second electrode surrounds a first electrode (viewed perpendicularly to skin contact regions corresponding to the electrodes);

FIG. 5B depicts a variation on the arrangement of FIG. 5B in which the second electrodes of the pairs of electrodes of FIG. 5A are joined together to form a common second electrode (viewed perpendicularly to skin contact regions corresponding to the electrodes);

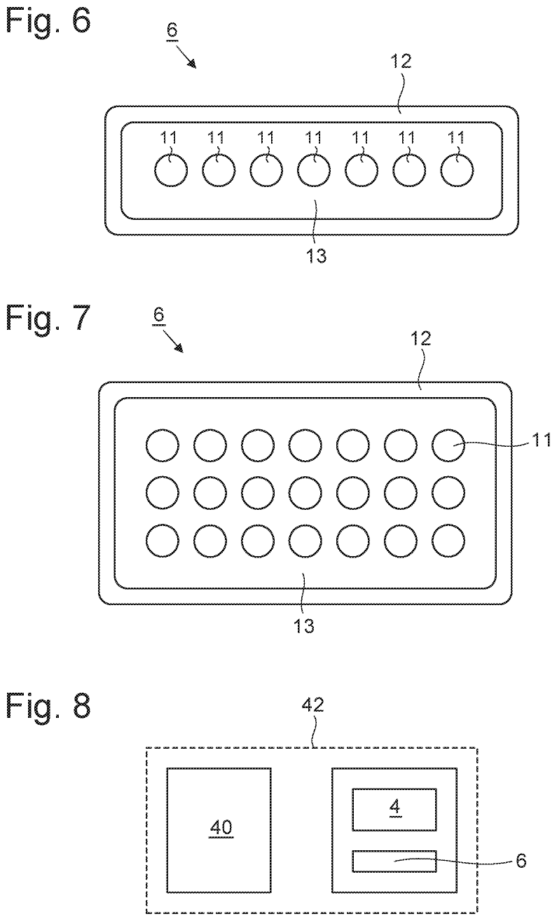

FIG. 6 depicts an example electrode unit comprising a row of first electrodes surrounding by a single second electrode (viewed perpendicularly to skin contact regions corresponding to the electrodes);

FIG. 7 depicts a further example electrode unit comprising plural rows of first electrodes surrounded by a single second electrode (viewed perpendicularly to skin contact regions corresponding to the electrodes);

FIG. 8 depicts an automated resuscitation system incorporating a system for electrically stimulating phrenic nerves to cause contraction of the diaphragm;

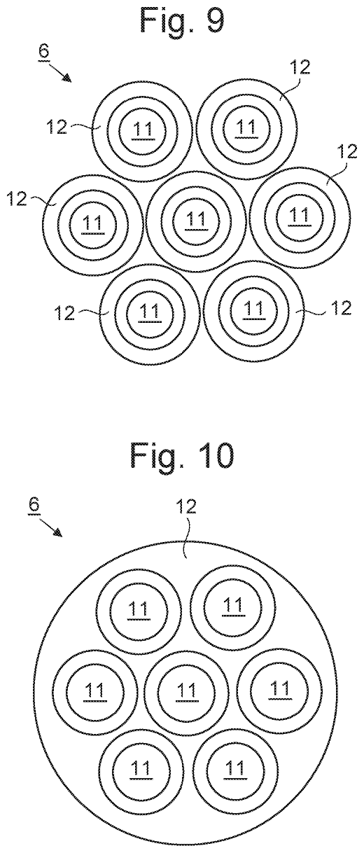

FIG. 9 depicts an alternative geometry for electrodes of the type depicted in FIG. 5A;

FIG. 10 depicts an alternative geometry for an arrangement of the type depicted in FIG. 5B;

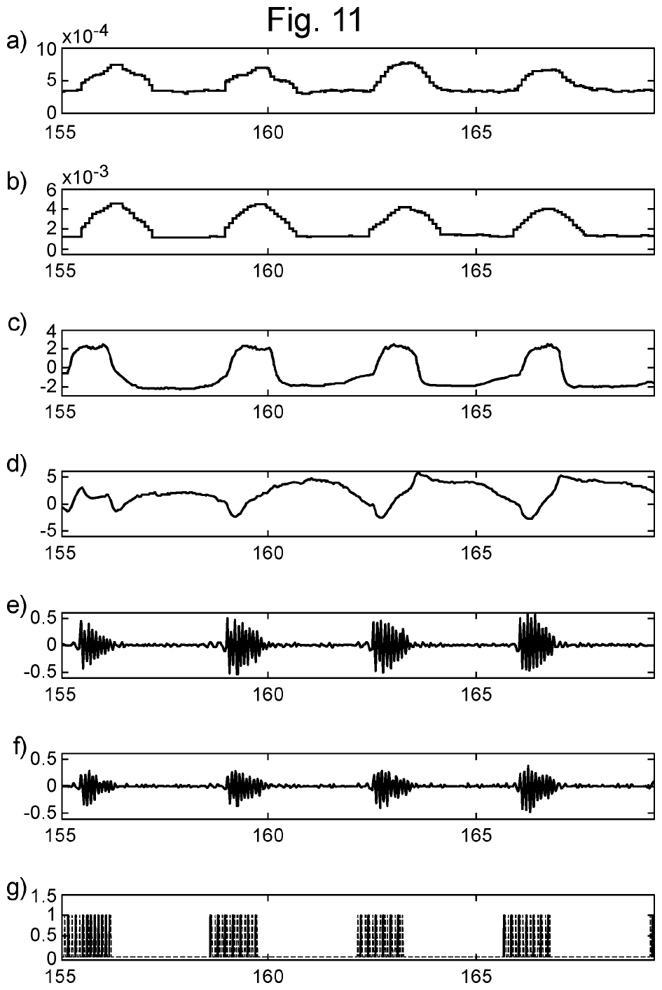

FIG. 11 depicts graphs showing results obtained during functional testing in an experimental demonstration of non-invasive phrenic nerve stimulation (Project A);

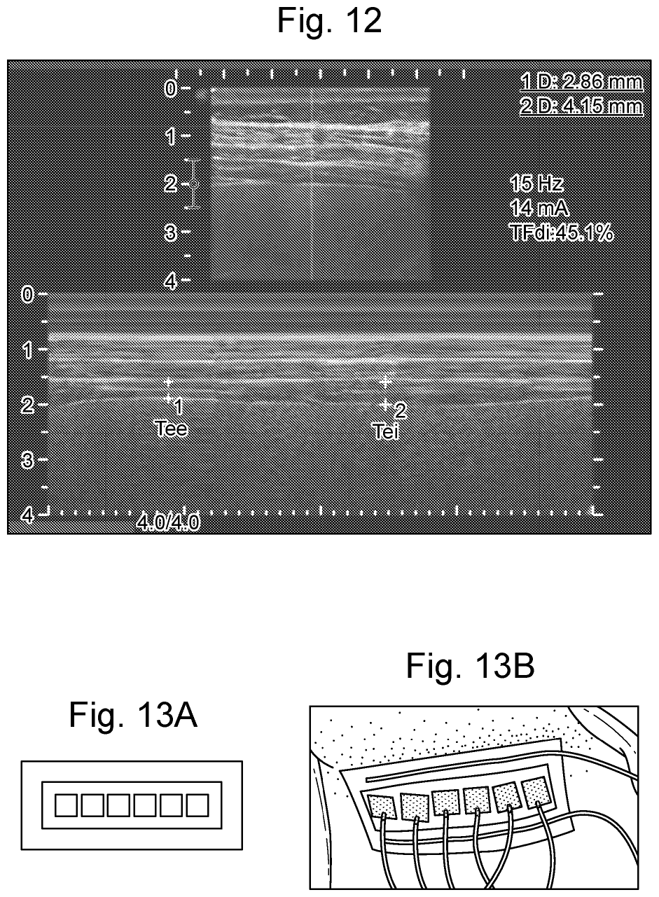

FIG. 12 depicts representative diaphragm thickening fraction (TF_{di}), assessed by ultrasound, in response to electrical stimulation;

FIG. 13 depicts an electrode configuration used in an experimental demonstration (Project B)-(A) schematic of configuration/(B) photograph of configuration in situ;

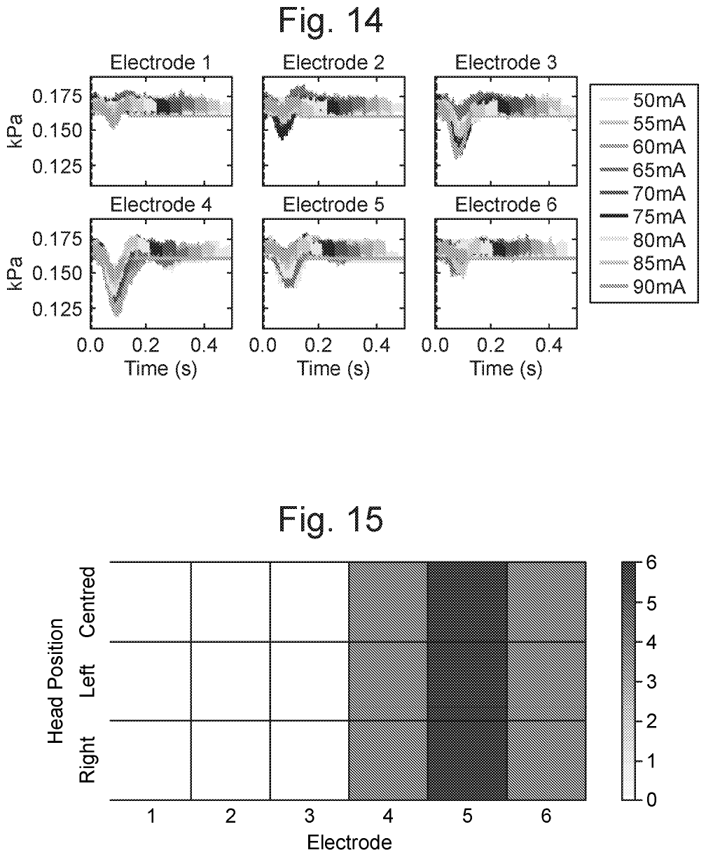

FIG. 14 depicts graphs showing example differential pressure generated by stimulation at each electrode over a range of amplitudes in Project B, showing that response is sensitive to electrode positioning, with a rapid drop off in response between the optimal electrode and its neighbour;

FIG. 15 is a heatmap of optimal electrode position across participants, with electrode 1 being most medial and electrode 6 most lateral; all participants responded to one of electrodes 4-6 and the optimal electrode did not change with head position;

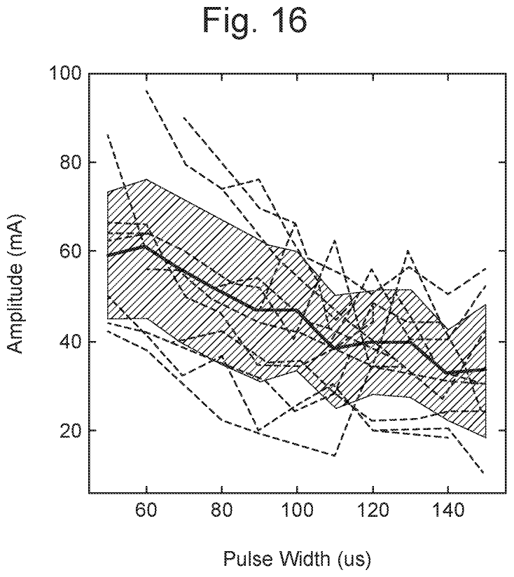

FIG. 16 depicts a graph showing a strength-duration curve, with the mean amplitude threshold across pulse widths (n=14) for single pulses shown; the standard deviation is shown as the shaded region; the individual participants' strength-duration curves are shown as dotted lines; the amplitude required to induce diaphragm contraction decreases as pulse width increases;

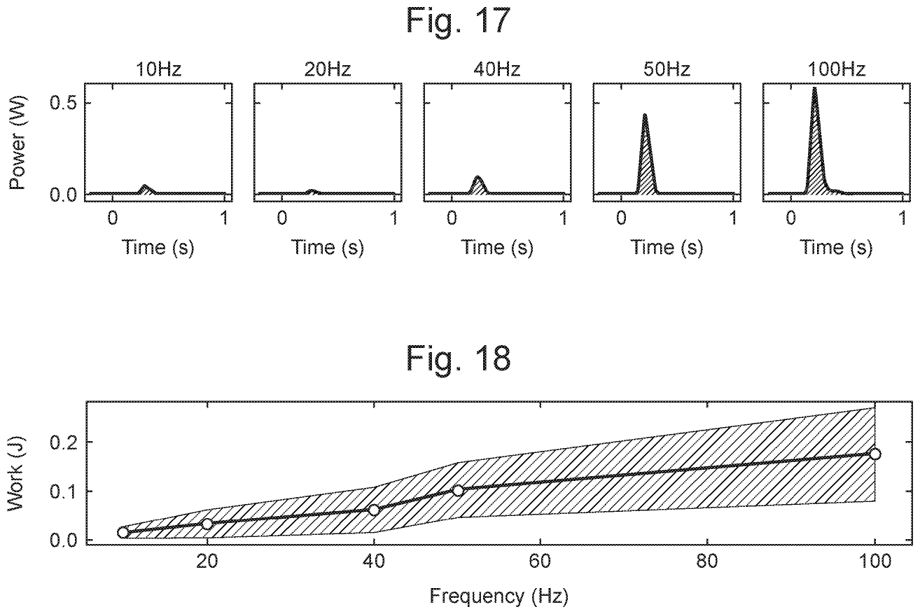

FIG. 17 depicts graphs showing example diaphragm activity (W) following pulse train at a range of frequencies; and

FIG. 18 depicts diaphragmatic work done (J) following stimulation for a range of frequencies; mean work done is shown; standard deviation is shown as the shaded region; increasing stimulation frequency induces greater diaphragmatic work.

As exemplified in FIG. 1, the present disclosure relates to systems for electrically stimulating a nerve to cause muscle contraction. The system comprises a plurality of electrodes 11, 12. Example configurations for the electrodes 11, 12 will be discussed in further detail below with reference to FIGS. 4-7. The electrodes 11, 12 are used to provide test stimulations and therapy stimulations. The electrodes 11, 12 may be provided in one or a plurality of electrode units 6. Where plural electrode units 6 are provided, each electrode unit 6 may comprise a respective subset of the plurality of electrodes 11, 12. Each electrode unit 6 may be positionable on the skin independently of each other electrode unit 6. The electrode units 6 may thus be moveable relative to each other, for example as mechanically separate elements or as elements that are connected by a deformable member. In some arrangements, the electrodes 11, 12 within each electrode unit 6 have a fixed spatial relationship with respect to each other. The provision of plural electrode units 6 provides flexibility for covering an area of interest on the skin. In the example of FIG. 1, two electrode units 6 are provided that can be positioned on left and right portions of the neck for stimulation of different phrenic nerves. Stimulation of the left and right phrenic nerves may be achieved simultaneously via respective combinations of electrodes in each of the two electrode units 6 (e.g. the left electrode unit 6 stimulates the left phrenic nerve and the right electrode unit 6 stimulates the right phrenic nerve). The electrode units 6 may preferably be elongate to ensure efficient coverage of a region of interest. The selection of the one or subset of the stimulation regions by the controller 4 may comprise selecting one or a subset of stimulation regions for each of the electrode units 6.

The electrodes 11, 12 are configured to be positioned in electrical contact with the skin simultaneously. The electrodes 11, 12 allow electrical stimulation to be directed independently towards a plurality of different stimulation regions below the skin via a corresponding plurality of combinations of the electrodes. Thus, different combinations of the electrodes 11, 12 allow targeting of different stimulation regions. Each accessible stimulation region has a unique combination of electrodes 11, 12 associated with it. Each combination of electrodes 11, 12 may typically comprise two electrodes (which may be referred to as an anode and cathode for example). However, it is also possible for more than two electrodes to be used to target one stimulation region, for example by using interference between different electrical signals.

The system comprises a controller 4. The controller 4 may comprise any suitable combination of data processing hardware, firmware and/or software, electronics, power supply hardware, and/or sensing apparatus necessary to provide the functionality described.

The controller 4 is configured to apply a test stimulation using each combination of electrodes 11, 12 and measure a respective response to the test stimulation (e.g. via data input 8 from one or more sensors, such as airflow sensor 10 described below and/or EMG, MMG and/or acoustic sensors). Thus, if five different combinations of electrodes 11, 12 are to be used the controller 4 will apply five test stimulations consisting of one test stimulation for each of the different combinations of electrodes 11, 12. The controller 4 selects one or a subset of stimulation regions based on the measured responses. The controller 4 applies a therapy stimulation to each selected stimulation region. Each therapy stimulation is applied using the combination of electrodes 11, 12 corresponding to the selected stimulation region. In the case where two or more stimulation regions are selected, the therapy stimulation is directed to each region using the combination of electrodes 11, 12 corresponding to that region (which will be different for each region).

Each measured response comprises information about a physiological response to the corresponding test stimulation. Each test stimulation can thus measure how effective the corresponding combination of electrodes would be in stimulating a desired physiological response in the patient. The test stimulations thus make it possible to select the most appropriate combination or combinations of electrodes to use to provide the therapy stimulation or therapy stimulations.

In some arrangements, the system is used to stimulate the phrenic nerve or nerves to cause contraction of the diaphragm. In these applications, the physiological response that is measured for each test stimulation may be a physiological response of the diaphragm, measured for example by a wearable sensor module. In some arrangements, the measured response may comprise one or more surface electromyographic (EMG) m-waves. Alternatively or additionally, the measured response may comprise a measure of a change in airway flow or pressure caused by the test stimulation (a larger or faster change in flow or drop in pressure being associated with a larger response of the diaphragm to the test stimulation). The change in airway flow or pressure may be obtained from an airflow sensor 10 connected to an airflow conduit associated with a mechanical ventilation system being used to apply assisted ventilation to the subject. Alternatively or additionally, the measured response may be mechanical, such as one or more mechanomyograms (MMGs). The MMGs may be measured using an accelerometer (implemented for example as a MEMS) or a microphone. The accelerometer may be positioned on the skin to detect the diaphragm twitching.

In some arrangements, the measured response may comprise one or more acoustic signals. The acoustic signals may be derived from tracheal or airway sounds. The acoustic signals may be measured for example by a microphone. The microphone may be positioned on the patient's thorax or on an intra tracheal tube or tubing to a ventilator. The acoustic signals may be processed digitally to provide a control signal. In the present application, the control signal may be used to select the most appropriate combination or combinations of electrodes to use to provide the therapy stimulation or therapy stimulations. Acoustic signals may, however, be used for other purposes, such as for synchronizing stimulation with ventilation (discussed further below), for assessing diaphragm condition (e.g. by providing information about a mechanical response of the diaphragm to a therapy stimulation), or for obtaining other physiologically relevant information, such as detection of lung sounds that may signal a need for intervention or care, such as suction. Using sound as a source of feedback is attractive because it requires no adhesive to the skin and microphones are physically small and low cost. The acoustic signal is furthermore relatively wideband, which enables fast response to transients. The acoustic signal may thus be easier to implement than EMG for diaphragm assessment and rapid electrode selection, and/or easier to implement than ECG for breathing state synchronization between the ventilator and stimulator.

In some arrangements signals from different sensors (e.g. EMG, MMG, air flow/pressure, and/or acoustic) may be combined, using processing techniques, to further improve the assessment of the physiological status of the diaphragm. The initial setting for the test pulses may be determined by the method of Mogyoros (“Strength-duration properties of human peripheral nerve”; Brain 1996, 119:439-447), for example to determine the pulse duration and current that produces a measurable diaphragmatic EMG, MMG and/or pressure/flow response with minimum charge delivery.

In some arrangements, the selection of the one or subset of the stimulation regions by the controller 4 comprises selecting the stimulation region for which the measured response to the respective test stimulation is the highest. Where plural electrode units 6 are provided, the selection may comprise selecting the stimulation region for which the measured response is highest for each of the electrode units 6. For example, in the arrangement of FIG. 1, one stimulation region may be selected for the electrode unit 6 on the left side of the neck (e.g. the one corresponding to the largest response to the test simulation for that electrode unit 6) and another stimulation region may be selected for the electrode unit 6 on the right side of the neck (e.g. the one corresponding to the largest response to the test simulation for that electrode unit 6).

In some arrangements, the selection of the one or subset of the stimulation regions by the controller 4 is performed based on whether the respective measured response is, or the respective measured responses are, higher than a predetermined response threshold. The test simulations may thus be used to detect combinations of electrodes that produce satisfactory responses. Electrodes to use in the therapy stimulation may then be selected from a set of combinations of electrodes determined to produce satisfactory responses.

In some arrangements, the controller 4 is further configured to perform a contact quality check. The contact quality check comprises measuring an electrical impedance associated with a contact between an electrode and the skin for each of the plurality of combinations of the electrodes. The controller 4 may output an alert (e.g. as a visual or audible signal, or as a data output to a separate device) when the measured electrical impedance for one or more of the contacts is higher than a predetermined impedance threshold. The contact quality check may thus detect failure (e.g. detachment) or aging or deterioration due to the state of interface gel hydration of individual electrodes. In some arrangements, a stimulation region may be excluded from the selection of the one or subset of stimulation regions when the measured electrical impedance for one or more of the contacts of the combination of electrodes corresponding to that stimulation region is higher than a predetermined impedance threshold. Thus the system can avoid applying the therapy using combinations of electrodes that includes a poor electrical contact with the skin, and thus avoid injecting a fixed charge through a small area of skin which can cause damage to the skin.

In some arrangements, the therapy stimulations are applied at intervals separated by one or more breathing cycle periods. The intervals may be regular or irregular. In some arrangements, the intervals are evenly spaced throughout the day. Arrangements of the present disclosure make such an approach practical because there is less or no need for manual supervision of the stimulation procedure. Spreading the stimulation more evenly through the day may help promote efficacy and/or reduce stress on the patient. It is commonly thought that about 200 stimulated contractions a day is effective to reduce diaphragm wasting. Thus, the therapy stimulation may be provided, for example, every 7-8 minutes. Each application of the therapy stimulation may thus typically be separated by many breathing cycles (e.g. 100-150).

In some arrangements, a separate instance of the test stimulation is performed before each application of the therapy stimulation. The selection of the one or subset of the stimulation regions for each therapy stimulation may thus be based on measured responses from test stimulations applied later than a most recent preceding application of the therapy stimulations. Effects from changes in the subject, such as changes in a head or torso position, can be taken into account and corrected for (e.g. by selecting a different electrode combination for therapy stimulation). In some arrangements, the system is additionally configured to perform a contact quality check before each application of the therapy stimulation. As described above, the contact quality check identifies contacts that may have deteriorated in the interval since the last therapy stimulation and can be used to ensure that the therapy stimulation is not applied using poor quality contacts. In some embodiments, each contact quality check is performed based on a corresponding test stimulation. Voltage and current measured during the test stimulation may be used to obtain an impedance value that is dependent on contact impedances of the electrodes involved. When one of these contacts is poor the overall impedance will become very large, thereby indicating the presence of a poor contact with possibility of tissue damage due to higher skin current density.

The test simulation may take any form that generates a suitable physiological response (e.g. a response that is strong enough to provide useful information about how effective the corresponding electrode combination would be for applying a therapy stimulation). It is the charge per pulse that causes the effect. Charge is the product of pulse width and current (in constant current stimulation). Various approaches can be used to provide as suitable pulse. A low-cost stimulator may be implemented as a battery powered systems with transformer coupled outputs. The range of output currents can be increased by increasing pulse width, until the transformer saturates. Higher pulse widths can be generated with high frequency switched-mode power supply techniques where the charge is generated at high frequency and stored on a capacitor until required in a stimulus pulse.

The test stimulation may comprise one or a plurality of test waveforms. Each test waveform may have a phase width 26 in the range of 50 us to 2 ms. The test waveform may have a peak current amplitude in the range of 1 mA to 200 mA. In implementations where the test stimulation comprises a plurality of test waveforms, at least a subset of the test waveforms may have different peak current amplitudes. In some arrangements, as exemplified schematically in FIG. 2, the test waveform 20 is biphasic, preferably comprising or consisting of a biphasic rectangular wave. Biphasic waveforms reduce or avoid net injection of charge, which may cause tissue damage in the long term, including for example skin damage due to changes in pH of interface gel. The biphasic waveform may comprise a first portion 21 having a first polarity (e.g. with a first electrode of a corresponding combination of electrodes having a higher potential than a second electrode of the combination) and a second portion 22 have a second polarity opposite to the first polarity (e.g. with the first electrode having a lower potential than the second electrode). The biphasic waveform may be charge balanced. An equal and opposite amount of charge may thus flow during each of the first and second portions 21, 22.

Test stimulations using each of the different combinations of electrodes will typically be applied at different times to make it possible to reliably identify which measured response corresponds to which combination of electrodes. The test stimulation may, for example, be applied using each of the combinations of electrodes one after the other (i.e., sequentially). The test stimulations may be spaced apart from each other in time to an extent that is sufficient to ensure that a physiological response from one test stimulation does not significantly interfere with the physiological response from any other test stimulation, for example by around a time period in the range of 10 ms-1 min. In some implementations, a cycle of sequential application of the test simulations to all of the different combinations of electrodes may be repeated to implement a plurality of cycles of test stimulation. Each cycle may use different test stimulations. For example, a peak current amplitude of test waveforms may be gradually increased from one cycle to the next cycle until a satisfactory response is obtained.

In some arrangements, as exemplified in FIG. 3, each application of the therapy stimulation comprises applying a plurality of therapy waveforms. Only three of the therapy waveforms are shown in FIG. 3 for clarity. The overall therapy stimulation may comprise many more than three of the therapy waveforms. The therapy waveforms may be identical to each other or different. In some arrangements, the therapy waveforms are evenly separated from each other in time. In some arrangements, each therapy waveform is biphasic, preferably comprising or consisting of a biphasic square wave. A mean repetition rate (1 divided by the period 34 indicated in FIG. 3) of the therapy waveforms may be in the range of 5-80 Hz. The period 34 may be constant or may vary (e.g. to provide a ramped repetition rate that provides a softer onset of contraction). The duration of the therapy stimulation (i.e. the length of the smallest time window encompassing all of the therapy waveforms) may be in the range of 0.5 s to 2 s.

In some arrangements, each of the therapy waveforms is the same as each of the test waveforms.

In some arrangements, the therapy waveforms have a phase width 36 in the range of 50 us to 2 ms. The therapy stimulation may comprise therapy waveforms that each have a peak current magnitude in the range of 1 mA to 200 mA.

In some arrangements, the onset and duration of the therapy stimulation is synchronized with the breathing cycle of the subject. The therapy stimulation may for example be applied exclusively within a predetermined portion of the breathing cycle. The predetermined portion may be selected to minimize interference with assisted ventilation (e.g. mechanical assisted ventilation provided by an external apparatus) or to have the diaphragm work against the assisted ventilation for a portion of the cycle to provide a form of resistance training. In some arrangements, a majority of the predetermined portion of the breathing cycle is in the inspiration phase of the assisted ventilation. Applying the therapy stimulation in the inspiration phase ensures that muscle contraction caused by the stimulation works in phase with the ventilation process, thereby reducing the size of unwanted pressure changes in the lungs. In some arrangements, all or a majority of the predetermined portion of the breathing cycle is in the first half of the inspiration phase. Applying the therapy stimulation at the start of the inspiration phase helps to ensure that the therapy simulation takes place predominantly or entirely within the inspiration phase even in the case where there are variations in the ventilation rate or timing, or where the duration of the therapy stimulation is relatively large in comparison with the ventilation period. It also ensures that the diaphragm contracts from a lengthened condition which may optimise the stimulus to reduce wasting.

The duration of the therapeutic stimulus may also be calculated as a percentage of the inspiratory time or respiratory cycle, since the instantaneous respiratory rate will be constantly monitored as described below.

The timing of the breathing cycle for performing the synchronization may be determined in various ways. In one arrangement, the timing is obtained at least partially from an air flow sensor. The air flow sensor may be incorporated into a conduit inserted into the subject's mouth as part of an assisted ventilation system, for example. The air flow sensor provides data indicative of flow into or out of the subject's lungs. The data may be derived from a measurement of a pressure differential along the conduit for example. Alternatively or additionally, the timing for performing the synchronization may be obtained at least partially from electrocardiogram (ECG) data. The electrocardiogram data may be obtained using the electrodes used to apply the test and/or therapy stimulations. Alternatively, further electrodes may be used to obtain the ECG data. Alternatively or additionally, as discussed above, the timing for performing the synchronization may be obtained at least partially from an acoustic signal that is sensitive to the breathing cycle.

Exemplary arrangements for implementing the electrodes that apply the test and therapy stimulations are now discussed with reference to FIGS. 4-7, which depict electrodes viewed perpendicularly to skin contact regions corresponding to the electrodes. The shapes of the electrodes shown in FIGS. 4-7 thus indicate the shapes of respective skin contact regions.

In some arrangements, the plurality of electrodes 11, 12 comprise a plurality of first electrodes 11. The first electrodes 11 are configured to be brought simultaneously into contact with respective different locations on the skin. Each location may be spaced apart from each other location. In the example of FIGS. 4, 5A and 5B, three first electrodes 11 are provided. The first electrodes 11 are evenly spaced along a horizontal direction in these examples. In FIG. 6, a row of seven first electrodes 11 is provided. In FIG. 7, three rows of seven first electrodes 11 are provided. Various other configurations may be provided. The optimum configuration may depend on the size and shape of the skin location where the first electrodes 11 are needed, as well as the degree of selectable spatial granularity that is desired for the therapy.

The system further comprises one or more second electrodes 12. The second electrodes 12 contact one or more respective further locations on the skin and have a different polarity to the first electrodes 11 during operation. Each of the combinations of electrodes 11, 12 corresponding to a stimulation region includes at least one of the first electrodes 11 and one of the second electrodes 12. The first and second electrodes 11, 12 may be mechanically connected to each other via an electrically insulating member 13.

In the example of FIG. 4, first and second electrodes 11, 12 are provided in bipolar pairs. Each bipolar pair constitutes one combination of electrodes that can direct an electrical stimulation to a respective stimulation region in the body below the portion of skin that the electrodes contact. In the example shown, the first and second electrodes 11, 12 are identical to each other in each pair (i.e. they have the same size and shape). The first and second electrodes 11, 12 are circular in the example shown but other shapes could be used. Any of two or more (or all) of the first and second electrodes 11, 12 may be mechanically connected to each other via an electrically insulated member 13, although this is not shown in FIG. 4.

In the example of FIG. 5A, first and second electrodes 11, 12 are provided in bipolar pairs in which a single second electrode 12 surrounds a single respective first electrode 11. Each of the second electrodes 12 are separate elements. A further example of this type of arrangement is shown in FIG. 9, where the bipolar pairs are arranged in a hexagonal grid. In the example of FIG. 5B, the second electrodes are effectively connected together to form a single common second electrode 12. The same second electrode 12 thus surrounds multiple different first electrodes 11, although the first electrodes 11 are separated from each other by a portion of the second electrode 12 extended between them. A further example of this type of arrangement is shown in FIG. 10, where the multiple first electrodes 11 are arranged in a hexagonal grid. The arrangements of FIGS. 5A and 5B (and FIGS. 9 and 10) are thus examples of arranging the plurality of electrodes so that a skin contact region of each of one or more of the second electrodes 12 surrounds a skin contact region of one or more of the first electrodes 11. Relative to the arrangement of FIG. 4, arranging for the second electrode 12 to surround the first electrode 11 provides a more focused and/or deeper stimulation.

In the examples of FIGS. 5A and 9, each second electrode 12 surrounds one and only one first electrode 11. This is not essential however. In other arrangements, the skin contact region of each of one or more of the second electrodes 12 may surround the skin contact region of multiple first electrodes 11. Example arrangements are shown in FIGS. 5B, 6, 7 and 10. These configurations, particularly in the case of arrangements of the type depicted in FIGS. 6 and 7 (where at least one of the second electrodes 12 is such that the skin contact regions of at least two of the first electrodes 11 surrounded by the skin contact region of the second electrode 12 are not separated from each other by a portion of the skin contact region of the second electrode 12), makes it easier to provide a high level of spatial granularity. Fine spatial separation between the centres of different stimulation regions can be achieved without sacrificing the desired spatial focusing effect provided by arranging for the first electrodes 11 to be surrounded by a second electrode 12.

In some arrangements, at least one of the combinations of electrodes comprises a plurality of at least the first electrodes. The controller may then drive that combination to provide the therapy stimulation by applying different respective electrical stimulations simultaneously via the plurality of first electrodes. The therapy stimulation may result from interference between the different respective electrical stimulations. For example, if time-modulated stimulations at different frequencies are applied simultaneously by the different first electrodes, beating may occur at a frequency equal to the difference between the two frequencies.

The plurality of electrodes may be configured be attached to the skin, for example using a skin-compatible adhesive.

FIG. 8 schematically depicts an automated resuscitation system 42 that uses arrangements of the present disclosure for automatically stimulating breathing in addition to the usual functionality associated with automatically applying shocks to the heart. The automated resuscitation system 42 may comprise an automated defibrillation unit 40 for applying shocks to the heart. The automated defibrillation unit 40 may be configured to measure an electrical output from a patient's heart and selectively deliver an electrical shock to the patient, dependent on the result of the measured electrical output, to restore normal heart function. Automated defibrillation units 40 are known in the art generally and any of the various known arrangements may be used for this aspect of the automated resuscitation system 42. In contrast to known automatic resuscitation systems, in the present arrangement the automatic resuscitation system 42 further comprises a system for electrically stimulating a nerve according to any of the arrangements disclosed herein. The system for electrically stimulating a nerve may comprise a controller 4 and one or more electrode units 6, as described above. The system for electrically stimulating a nerve may be configured particularly to stimulate the phrenic nerves in order to cause contraction of the diaphragm and thereby initiate or assist breathing in a patient.

Thus, a method may be provided that comprises positioning a plurality of electrodes in electrical contact with the skin of a subject simultaneously. The electrodes are configured to allow electrical stimulation to be directed independently towards a plurality of different stimulation regions below the skin via a corresponding plurality of combinations of the electrodes. For each combination of electrodes, a test stimulation is applied using the combination of electrodes and a response to the test stimulation is measured. One or a subset of stimulation regions is/are selected based on the measured responses. A therapy stimulation is applied to each selected stimulation region using the combination of electrodes corresponding to the selected stimulation region.

A method may further be provided that comprises positioning a plurality of electrodes in electrical contact with the skin of a subject simultaneously. The electrodes are configured to allow electrical stimulation to be directed independently towards a plurality of different stimulation regions below the skin via a corresponding plurality of combinations of electrodes. The plurality of electrodes comprises a plurality of first electrodes. The first electrodes are simultaneously in contact with respective different locations on the skin. The plurality of electrodes further comprises one or more second electrodes contacting one or more respective further locations on the skin. A skin contact region of each of one or more of the second electrodes surrounds a skin contact region of a plurality of the first electrodes. The electrodes are operated (e.g. to apply test stimulations and/or therapy stimulations) with the one or more second electrodes having different polarity to the first electrodes.

Embodiments are defined in the following numbered clauses.
1. A system for electrically stimulating a nerve to cause muscle contraction, comprising:
   a plurality of electrodes configured to be positioned in electrical contact with the skin simultaneously, the electrodes being configured to allow electrical stimulation to be directed independently towards a plurality of different stimulation regions below the skin via a corresponding plurality of combinations of the electrodes; and
   a controller configured to:
   for each combination of electrodes, apply a test stimulation using the combination of electrodes and measure a response to the test stimulation;
   select one or a subset of stimulation regions based on the measured responses; and
   apply a therapy stimulation to each selected stimulation region using the combination of electrodes corresponding to the selected stimulation region.
2. The system of clause 1, wherein each measured response comprises information about a physiological response to the corresponding test stimulation.
3. The system of clause 2, wherein the physiological response comprises a physiological response of the diaphragm.
4. The system of clause 2 or 3, wherein the measured response comprises one or more of the following: a surface electromyographic m-wave; a mechanomyogram; an acoustic signal.
5. The system of clause 3 or 4, wherein the measured response comprises a measure of a change in airway flow or pressure caused by the test stimulation.
6. The system of any preceding clause, wherein the controller is configured such that the selection of the one or subset of the stimulation regions comprises selecting the stimulation region for which the measured response to the respective test stimulation is the highest.
7. The system of any preceding clause, wherein the controller is configured such that the selection of the one or subset of the stimulation regions is performed based on whether the respective measured response is, or respective measured responses are, higher than a predetermined response threshold.
8. The system of any preceding clause, wherein the electrodes are provided as a plurality of electrode units, each electrode unit comprising a respective subset of the plurality of electrodes.
9. The system of clause 8, wherein each electrode unit is moveable relative to the other electrode unit.
10. The system of clause 8 or 9, wherein the controller is configured such that the selection of the one or subset of the stimulation regions comprises selecting one or a subset of stimulation regions for each of the electrode units.
11. The system of any preceding clause, wherein the controller is further configured to perform a contact quality check comprising measuring an electrical impedance associated with a contact between an electrode and the skin for each of the plurality of combinations of the electrodes.
12. The system of clause 11, wherein the controller is configured to output an alert when the measured electrical impedance for one or more of the contacts is higher than a predetermined impedance threshold.
13. The system of clause 11 or 12, wherein the controller is configured to exclude a stimulation region from the selection when the measured electrical impedance for one or more of the contacts of the combination of electrodes corresponding to that stimulation region is higher than a predetermined impedance threshold.
14. The system of any preceding clause, wherein:
   the therapy stimulations are applied at intervals separated by one or more breathing cycle periods; and
   a separate instance of the test stimulation is performed before each application of the therapy stimulation.
15. The system of clause 14, wherein a separate instance of a contact quality check is performed before each application of the therapy stimulation.
16. The system of clause 14 or 15, wherein the controller is configured such that the selection of the one or subset of the stimulation regions for each therapy stimulation is based on the measured responses from test stimulations applied later than a most recent preceding application of the therapy stimulations.
17. The system of any preceding clause, wherein each test stimulation comprises a biphasic waveform, preferably a biphasic rectangular wave.
18. The system of any preceding clause, wherein the test stimulation is applied using each of the combinations of electrodes one after another.
19. The system of any preceding clause, wherein each application of the therapy stimulation comprises applying a plurality of therapy waveforms.
20. The system of clause 19, wherein the therapy stimulation satisfies each of one or more of the following in any combination:
   a duration of the therapy stimulation is in the range of 0.5 s to 2 s;
   each therapy waveform is biphasic, preferably a biphasic rectangular wave;
   a peak current amplitude in each therapy waveform is in the range of 1-200 mA;
   a phase width of each therapy waveform is in the range of 50 μs to 2 ms.
21. The system of any preceding clause, wherein the controller is further configured to synchronize the application of the therapy stimulation with the breathing cycle.
22. The system of clause 21, wherein the controller is configured to apply the therapy stimulation only within a predetermined portion of the breathing cycle of a subject.
23. The system of clause 22, wherein the predetermined portion of the breathing cycle is selected to minimize interference with assisted ventilation applied to the subject.
24. The system of clause 22 or 23, wherein a majority of the predetermined portion of the breathing cycle is in the inspiration phase of assisted ventilation applied to the subject.
25. The system of any of clauses 22-24, wherein a majority of the predetermined portion of the breathing cycle is in the first half of the inspiration phase of assisted ventilation applied to the subject.
26. The system of any of clauses 21-25, wherein the controller is configured to determine a timing of the breathing cycle, for performing the synchronization, at least partially from an air flow sensor.
27. The system of any of clauses 21-26, wherein the controller is configured to determine a timing of the breathing cycle, for performing the synchronization, at least partially from electrocardiogram data.
28. The system of any of clauses 21-27, wherein the controller is configured to determine a timing of the breathing cycle, for performing the synchronization, at least partially from an acoustic signal.
29. The system of any preceding clause, wherein:
   the plurality of electrodes comprises a plurality of first electrodes configured to be brought simultaneously into contact with respective different locations on the skin; and
   one or more second electrodes configured to contact one or more respective further locations on the skin, and to have different polarity to the first electrodes during operation of the system.
30. The system of clause 29, wherein each combination of electrodes corresponding to a stimulation region includes at least one of the first electrodes and one of the second electrodes.
31. The system of clause 29 or 30, wherein the plurality of electrodes is configured such that a skin contact region of each of one or more of the second electrodes surrounds a skin contact region of one or more of the first electrodes.
32. The system of clause 31, wherein the plurality of electrodes is configured such that the skin contact region of each of one or more of the second electrodes surrounds the skin contact region of multiple first electrodes, preferably with at least one of the second electrodes being configured such that the skin contact regions of at least two of the first electrodes surrounded by the skin contact region of the second electrode are not separated from each other by a portion of the skin contact region of the second electrode.
33. The system of any of clauses 29-32, wherein at least one of the combinations of electrodes comprises a plurality of at least the first electrodes, and the controller is configured to drive that combination to provide the therapy stimulation by applying different respective electrical stimulations simultaneously via the plurality of first electrodes, the therapy stimulation resulting from interference between the different respective electrical stimulations.
34. The system of any preceding clause, wherein the plurality of electrodes are configured to be attached to the skin, preferably adhesively.
35 An automated resuscitation system, comprising:
   an automated defibrillation unit configured to measure an electrical output from a patient's heart and selectively deliver an electrical shock to the patient to restore normal heart function; and
   the system of any preceding clause configured to stimulate contraction of the diaphragm to restore normal breathing function.
36. A system for electrically stimulating a nerve to cause muscle contraction, comprising:
   a plurality of electrodes configured to be positioned in electrical contact with the skin simultaneously, the electrodes being configured to allow electrical stimulation to be directed independently towards a plurality of different stimulation regions below the skin via a corresponding plurality of combinations of electrodes, wherein the plurality of electrodes comprises:
   a plurality of first electrodes configured to be brought simultaneously into contact with respective different locations on the skin; and
   one or more second electrodes configured to contact one or more respective further locations on the skin, and to have different polarity to the first electrodes during operation of the system,
   wherein the plurality of electrodes is configured such that a skin contact region of each of one or more of the second electrodes surrounds a skin contact region of a plurality of the first electrodes.
37. A method of electrically stimulating a nerve to cause muscle contraction, comprising:
   positioning a plurality of electrodes in electrical contact with the skin of a subject simultaneously, the electrodes being configured to allow electrical stimulation to be directed independently towards a plurality of different stimulation regions below the skin via a corresponding plurality of combinations of the electrodes; and
   for each combination of electrodes, applying a test stimulation using the combination of electrodes and measuring a response to the test stimulation;
   selecting one or a subset of stimulation regions based on the measured responses; and
   applying a therapy stimulation to each selected stimulation region using the combination of electrodes corresponding to the selected stimulation region.
38. A method of electrically stimulating a nerve to cause muscle contraction, comprising:
   positioning a plurality of electrodes in electrical contact with the skin of a subject simultaneously, the electrodes being configured to allow electrical stimulation to be directed independently towards a plurality of different stimulation regions below the skin via a corresponding plurality of combinations of electrodes, wherein the plurality of electrodes comprises:
   a plurality of first electrodes simultaneously in contact with respective different locations on the skin; and
   one or more second electrodes contacting one or more respective further locations on the skin, a skin contact region of each of one or more of the second electrodes surrounding a skin contact region of a plurality of the first electrodes,
   operating the electrodes with the one or more second electrodes having different polarity to the first electrodes.
39. The method of clause 37 or 38, wherein the nerve is a phrenic nerve.

### Experimental Demonstration Showing Feasibility of Non-Invasive Phrenic Nerve Stimulation—Project A

### Methods

Healthy volunteers, all adults with no respiratory or neuro-muscular disorders, were recruited. Written informed consent was obtained from all subjects. The study was approved by the Ethics Committee at the Universidad de Concepción, Chile (CEBB 714-2020).

Stimulation system: A bespoke non-invasive electrical phrenic nerve stimulator was developed. The system comprised three parts: 1) Stimulator, 2) Monitoring, and 3) User interface. The stimulator was designed to produce a constant current monophasic waveform in two independent channels. Each channel can deliver current amplitudes from 1 mA to 150 mA into a 2 kOhm load with 1 mA step, frequencies from 1 Hz to 30 Hz with 1 Hz step, and pulse widths from 10 μs to 400 μs with 10 μs step. The monitoring system is able to measure diaphragm electromyography (EMG), mechanomyography (MMG), and airway flow and pressure signals. These parameters are used to evaluate subject's stimulation-response. Finally, the user interface was implemented on a touch screen, capable of modify stimulation parameters, and monitoring variables in real-time.
Intervention: To perform the tests, all volunteers were asked to lie on a clinical table. After that, vital signals (blood pressure, heart rate, respiration rate, oxygen saturation) were measured at the beginning, during, and at the end of the intervention. Next, monitoring electrodes (EMG, MMG), flow and pressure sensors were placed. A calibration process was necessary to find the motor point and define the proper stimulation electrode placement. In this process, a probe electrode (motor point pen) was used. As the probe electrode moved over the neck, typically posterior to the sternocleidomastoideole at the level of the cricoid cartilage, the current amplitude was modified until diaphragmatic activation was detected. The activation was corroborated by ultrasound to detect diaphragm movement, and through the monitoring sensors. Once the motor point was found, the stimulation electrodes were positioned. A concentric ring electrode arrangement was used comprising a circular central electrode acting as cathode and a concentric annular outer electrode acting as anode. These bespoke electrodes were made by modifying commercial electrodes (Axelgaard® Ultrastim); a 10 mm diameter was made for cathode and 40 mm and 30 mm external and internal diameter for the ring anode, respectively. Finally, volunteers were connected to a non-invasive mechanical ventilator (NIV). The ventilator was configured in assistive mode, and the ventilatory parameters were defined according to the volunteer's normal breathing rate. All volunteers were ventilated and electrically stimulated for 10 minutes.

### Results

10 healthy volunteers were evaluated, 5 male and 5 female. The mean (range) age was 31(26-39) years, the mean weight was 76.9 (66.1-94.6) kg., and mean BMI was 26.9 (22.9-36.6). Bilateral phrenic nerve stimulation was possible in all participants. Stimulation and ventilation were synchronised successfully. Vital signs measured during remain between physiological range, no significant changes were registered. 4 subjects reported discomfort during stimulation, mainly pricking sensations due to a lack in skin electrode adhesion. No other adverse effects were reported. Table 1 below shows stimulation parameters used during functional testing with the mechanical ventilator for each volunteer. The frequency values to sustained diaphragmatic contractions were achieved with frequencies between 11 Hz to 15 Hz. Concerning the current amplitude values, the mean value was 24 (14-38) mA for both nerves. The pulse widths had a mean value of 198 (150-280) μs.

TABLE 1
Electrical stimulation parameter used during functional testing

| | | | | | Right | Left |
|---|---|---|---|---|---|---|
| | | | | | phrenic | phrenic |
| | | | Fre- | Pulse | nerve | nerve |
| | | | quency | Width | amplitude | amplitude |
| Volunteer | Gender | Age | (Hz) | (us) | (mA) | (mA) |
| 1 | F | 33 | 15 | 200 | 14 | 14 |
| 2 | M | 39 | 14 | 200 | 26 | 26 |
| 3 | F | 33 | 13 | 180 | 29 | 29 |
| | F | 28 | 13 | 150 | 28 | 19 |
| 5 | F | 35 | 13 | 200 | 38 | 38 |
| 6 | M | 28 | 13 | 150 | 20 | 20 |
| 7 | M | 31 | 13 | 170 | 20 | 20 |
| | F | 29 | 15 | 280 | 22 | 25 |
| 9 | M | 26 | 11 | 250 | 20 | 20 |
| 10 | M | 28 | 14 | 200 | 25 | 25 |

FIG. 11 shows the results obtained during functional testing in one volunteer (stimulation parameters used were: 20 mA, 200 μs, and 13 Hz). Ventilator parameters used were respiratory rate of 18 bpm, inspiration/expiration ratio of 1:2, 0 cmH₂O of PEEP, and pressure support of 4 cmH20. Traces represents a) right and b) left integrated EMG; airway c) flow (L/min) and d) pressure (cmH₂O) signals; e) right f) left MMG (m/s²) signals; and g) stimulation pulse trains.

Representative diaphragm thickening fraction (TF_{di}), assessed by ultrasound, in response to electrical stimulation is shown in FIG. 12. Stimulation parameters used for this volunteer were: 14 mA, 200 μs, and 15 Hz, obtaining a TF_{di} of 45.1%.

### Discussion of Project A

Project A shows the feasibility of using a non-invasive phrenic nerve stimulator to activate the diaphragm during non-invasive ventilation. Phrenic nerve stimulation is seen to generate effective diaphragm contraction as a spontaneous breathing effort that trigger the ventilator and keeping safety ventilatory parameters, similar to clinical context in assistive ventilation.

### Experimental Demonstration Showing Feasibility of Non-Invasive Phrenic Nerve Stimulation—Project B

### Methods

Healthy volunteers with no respiratory or neurological pathologies were recruited. Electrodes were placed on the neck in the arrangement shown in FIG. 13. Six 1 cm×1 cm cathodes (Axelgaard®) were placed with 2 mm intervals and surrounded by an anode with a typical electrode impedance of 1470+/−14852. The medial edge of the electrode array was placed on the midline with the cathodes at the level of the cricoid cartilage.

Stimulation was applied using a Digitimer DSR8 current-regulated stimulator with a compliance voltage of 400V. Biphasic, charge-balanced, symmetric waveforms were used throughout. Each of the six cathodes was attached to a Digitimer D188 electrode selector to allow stimulation at individual electrode sites.

Measuring diaphragm activity: Respiratory responses to stimulation were recorded using pressure and flow sensors attached to a pitot tube section of an anaesthetic circuit which subjects breathed through, forming a seal with the lips around its end. Subjects maintained an open glottis and wore a nose clip to prevent escape of air through the nose. The work done by the diaphragm was then computed by calculating the pressure volume work over the period following stimulation, i.e. ∫P dV dt.
Assessing electrode position: The effect of electrode position was characterised by stimulating at each cathode sequentially at a range of amplitudes while the respiratory response was measured. This was performed with the head centred and then while looking to the left or to the right in order to assess the effect of head position on optimal electrode placement.
Assessing stimulation parameters: The electrode that induced the lowest threshold response was selected. The strength-duration curve for the phrenic nerve was then characterised by measuring the threshold required to produce a measurable diaphragm response for a range of pulse widths using single stimulation pulses.

In a subset of participants, the effect of stimulation frequency was investigated by delivering 200 ms pulse trains of varying frequency at the optimal electrode using 100 μs pulses at 125% of the threshold identified. The diaphragmatic pressure-volume work produced by stimulation was calculated and integrated over the 1 s following stimulation to measure the effect of stimulation.

### Results

14 participants were recruited (7 male, 7 female).

Electrode position: In all participants, one cathode showed a greater response than all the others, with a rapid drop in diaphragm response following stimulation at the neighbouring electrodes. An example of an electrode sweep over a range of amplitudes is shown in FIG. 14, where one electrode shows a clear response, while there is a smaller response at neighbouring electrodes and little response at other electrodes. The distributions of optimal electrodes across all participants for each head position are shown in FIG. 15. Most participants (6/14) had a maximal response at electrode 5 (68 mm from the midline), while 4 participants had a maximal response at the 4th (56 mm from the midline) and 6th (80 mm from the midline) electrodes. The optimal electrode did not vary with head position in any participant in this project.

Strength-duration relationship: Threshold amplitudes were variable across individuals. However, there was a decrease in threshold as pulse width increased in all participants. The mean strength-duration curve for the phrenic nerve using single stimulation pulses is shown in FIG. 16. This demonstrates a relative flattening of the threshold above a pulse width of 110 μs.
Stimulation frequency: 200 ms pulse trains of 100 μs pulses at 125% of the threshold identified on strength-duration testing produced a measurable diaphragm contraction. The work produced by stimulation increased as stimulation frequency increased. An example of the measured power (W) following pulse trains at varying frequencies is shown in FIG. 17. The total work (J) done during the induced contraction is shown in FIG. 18. This increases as stimulation frequency increases, with a sharp increase up to 50 Hz, and an average work at 100 Hz of 0.17+/−0.09 J, corresponding to an inhaled volume of 609+/−92 ml.

### Discussion of Project B

Project B further demonstrates that non-invasive stimulation of the phrenic nerve for inducing diaphragm contraction is feasible. Stimulation parameters for achieving reliable non-invasive activation of the nerve are characterised. The ability to activate the phrenic nerve non-invasively is shown to be sensitive to electrode position. Stimulation at the level of the cricoid cartilage reliably produced a response, but the lateral position required varied between individuals. All participants showed a response between 56 mm and 80 mm from the midline, with most responding at 68 mm, but the individual optimum position was variable.

Characterisation of the strength-duration relationship for non-invasive stimulation of the phrenic nerve and the relationship between stimulation frequency and inhaled volume provide a demonstration of the ability to achieve meaningful diaphragm contractions with non-invasive stimulation with parameters that are achievable with standard stimulation hardware.

1. A system for electrically stimulating a nerve to cause muscle contraction, comprising:
a plurality of electrodes configured to be positioned in electrical contact with the skin simultaneously, the electrodes being configured to allow electrical stimulation to be directed independently towards a plurality of different stimulation regions below the skin via a corresponding plurality of combinations of the electrodes; and
a controller configured to:
for each combination of electrodes, apply a test stimulation using the combination of electrodes and measure a response to the test stimulation;
select one or a subset of stimulation regions based on the measured responses; and
apply a therapy stimulation to each selected stimulation region using the combination of electrodes corresponding to the selected stimulation region.

2. The system of claim 1, wherein each measured response comprises information about a physiological response to the corresponding test stimulation.

3. The system of claim 2, wherein the physiological response comprises a physiological response of the diaphragm.

4. The system of claim 1, wherein the controller is configured such that the selection of the one or a subset of the stimulation regions comprises selecting the stimulation region for which the measured response to the respective test stimulation is the highest.

5. The system of claim 1, wherein the controller is configured such that the selection of the one or a subset of the stimulation regions is performed based on whether the respective measured response is, or respective measured responses are, higher than a predetermined response threshold.

6. The system of claim 1, wherein the controller is further configured to perform a contact quality check comprising measuring an electrical impedance associated with a contact between an electrode and the skin for each of the plurality of combinations of the electrodes.

7. The system of claim 6, wherein the controller is configured to output an alert or exclude a stimulation region from the selection when the measured electrical impedance for one or more of the contacts is higher than a predetermined impedance threshold.

8. The system of claim 1, wherein:
the therapy stimulations are applied at intervals separated by one or more breathing cycle periods; and
a separate instance of the test stimulation is performed before each application of the therapy stimulation.

9. The system of claim 1, wherein each test stimulation comprises a biphasic waveform, preferably a biphasic rectangular wave.

10. The system of claim 1, wherein the test stimulation is applied using each of the combinations of electrodes one after another.

11. The system of claim 1, wherein each application of the therapy stimulation comprises applying a plurality of therapy waveforms.

12. The system of claim 11, wherein the therapy stimulation satisfies each of one or more of the following in any combination:
a duration of the therapy stimulation is in the range of 0.5 s to 2 s:
each therapy waveform is biphasic, preferably a biphasic rectangular wave:
a peak current amplitude in each therapy waveform is in the range of 1-200 mA:
a phase width of each therapy waveform is in the range of 50 μs to 2 ms.

13. The system of claim 1, wherein the controller is further configured to synchronize the application of the therapy stimulation with the breathing cycle.

14. The system of claim 13, wherein the controller is configured to apply the therapy stimulation only within a predetermined portion of the breathing cycle of a subject.

15. The system of claim 14, wherein a majority of the predetermined portion of the breathing cycle is in the first half of the inspiration phase of assisted ventilation applied to the subject.

16. The system of Re-claim 1, wherein:
the plurality of electrodes comprises a plurality of first electrodes configured to be brought simultaneously into contact with respective different locations on the skin; and
one or more second electrodes configured to contact one or more respective further locations on the skin, and to have different polarity to the first electrodes during operation of the system.

17. The system of claim 16, wherein each combination of electrodes corresponding to a stimulation region includes at least one of the first electrodes and one of the second electrodes.

18. The system of claim 16, wherein the plurality of electrodes is configured such that a skin contact region of each of one or more of the second electrodes surrounds a skin contact region of one or more of the first electrodes.

19. The system of claim 18, wherein the plurality of electrodes is configured such that the skin contact region of each of one or more of the second electrodes surrounds the skin contact region of multiple first electrodes, preferably with at least one of the second electrodes being configured such that the skin contact regions of at least two of the first electrodes surrounded by the skin contact region of the second electrode are not separated from each other by a portion of the skin contact region of the second electrode.

20. The system of claim 16, wherein at least one of the combinations of electrodes comprises a plurality of at least the first electrodes, and the controller is configured to drive that combination to provide the therapy stimulation by applying different respective electrical stimulations simultaneously via the plurality of first electrodes, the therapy stimulation resulting from interference between the different respective electrical stimulations.

21. An automated resuscitation system, comprising:
an automated defibrillation unit configured to measure an electrical output from a patient's heart and selectively deliver an electrical shock to the patient to restore normal heart function; and
the system of claim 1 configured to stimulate contraction of the diaphragm to restore normal breathing function.

22. A system for electrically stimulating a nerve to cause muscle contraction, comprising:
a plurality of electrodes configured to be positioned in electrical contact with the skin simultaneously, the electrodes being configured to allow electrical stimulation to be directed independently towards a plurality of different stimulation regions below the skin via a corresponding plurality of combinations of electrodes, wherein the plurality of electrodes comprises:
a plurality of first electrodes configured to be brought simultaneously into contact with respective different locations on the skin; and
one or more second electrodes configured to contact one or more respective further locations on the skin, and to have different polarity to the first electrodes during operation of the system,
wherein the plurality of electrodes is configured such that a skin contact region of each of one or more of the second electrodes surrounds a skin contact region of a plurality of the first electrodes.

23. A method of electrically stimulating a nerve to cause muscle contraction, comprising:
positioning a plurality of electrodes in electrical contact with the skin of a subject simultaneously, the electrodes being configured to allow electrical stimulation to be directed independently towards a plurality of different stimulation regions below the skin via a corresponding plurality of combinations of the electrodes; and
for each combination of electrodes, applying a test stimulation using the combination of electrodes and measuring a response to the test stimulation;
selecting one or a subset of stimulation regions based on the measured responses; and
applying a therapy stimulation to each selected stimulation region using the combination of electrodes corresponding to the selected stimulation region.

24. A method of electrically stimulating a nerve to cause muscle contraction, comprising:
positioning a plurality of electrodes in electrical contact with the skin of a subject simultaneously, the electrodes being configured to allow electrical stimulation to be directed independently towards a plurality of different stimulation regions below the skin via a corresponding plurality of combinations of electrodes, wherein the plurality of electrodes comprises:
a plurality of first electrodes simultaneously in contact with respective different locations on the skin; and
one or more second electrodes contacting one or more respective further locations on the skin, a skin contact region of each of one or more of the second electrodes surrounding a skin contact region of a plurality of the first electrodes,
operating the electrodes with the one or more second electrodes having different polarity to the first electrodes.

25. The method of claim 23, wherein the nerve is a phrenic nerve.
